(19) United States
(12) Patent Application Publication
(10) Pub. No.: US 2013/0304081 A1
(43) Pub. Date: Nov. 14, 2013

(54) OBSTETRIC VACUUM EXTRACTOR

(71) Applicants: Samuel George, Weybridge Surrey (GB); Daniel Peterson Godfrey, Cambridge (GB); Lucy Ann Sheldon, Cambridge (GB); Ross William Weir, Cambridge (GB)

(72) Inventors: Samuel George, Weybridge Surrey (GB); Daniel Peterson Godfrey, Cambridge (GB); Lucy Ann Sheldon, Cambridge (GB); Ross William Weir, Cambridge (GB)

(73) Assignee: Monty Medical Limited, Redditch Worcestershire (GB)

(21) Appl. No.: 13/876,857

(22) PCT Filed: Sep. 29, 2011
(86) PCT No.: PCT/GB11/51850

§ 371 Date: Jun. 12, 2013

(30) Foreign Application Priority Data
Sep. 30, 2010 (GB) 1016466.3

(51) Int. Cl. A61B 17/44

(52) U.S. Cl. CPC A61B 17/442
USPC 606/123

(57) ABSTRACT

An obstetric vacuum extractor (20) comprises a cylinder (22) communicating with a cup (26), and a handle (46) connected to a piston (38) via an extensible bias element (52) to evacuate the cup. The bias element permits relative movement between the handle and the piston. The bias element limits force transmitted to the piston, and movement of the piston relative to the handle operates an indicator to confirm that low pressure has been maintained within the cup. A cup for such an extractor comprises inner (130) and outer (132) sections, at least the outer section being reconfigurable, by applying distal force to a proximal side of the cup, from a convex insertion state for insertion into a mother's birth canal into a concave engagement state for engagement with a baby's head. A boundary between the sections is shaped such that the inner section may be concave while the outer section is convex, to create an inner suction cup.

This invention relates to the field of obstetrics and particularly to apparatus for assisting in the delivery of babies.

Assisted delivery may be recommended if a baby becomes distressed or fails to make adequate progress through the birth canal during labour, or if the mother is unable to push due to tiredness or a medical condition and so needs help to expel the baby. Assisted delivery usually implies the use of either forceps or a vacuum extractor known generically as a ‘ventouse’.

Forceps typically have two intersecting metal parts with curved distal ends to cradle the baby's head. In use, the mother is placed on her back on a bed in the lithotomy position with her legs in stirrups. After being catheterised, she undergoes an episiotomy to enlarge the opening of her vagina so that the distal ends of the forceps can be put round the baby's head within her dilated cervix. Once the forceps are in place and locked, the attending physician pulls the proximal handle part of the forceps in time with the mother's contractions to help the baby progress through the birth canal.

In contrast, a ventouse comprises a vacuum cup communicating with a source of vacuum, such as a hand-held, foot-operated or electric vacuum pump connected to the cup by a short tube. It is also possible to connect the cup to a suction line leading to a remote vacuum source such as a vacuum reservoir system built in to a hospital. In use, with the mother in the lithotomy position as aforesaid, the cup is inserted into the vagina and oriented to fit on top of a baby's head, whereupon the cup is fixed to the baby's head by suction as the attending physician draws air out of the cup through the tube or suction line, causing the cup to seal round its distal periphery to the baby's scalp. A handle or strap attached proximally to the cup then enables force to be applied to the cup to manipulate the baby's position, the physician pulling the handle or strap in time with the mother's contractions to help the baby progress through the birth canal. The tube through which air is drawn from the cup may form part of the handle or it may effect attachment of the handle to the cup.

The cup is typically made of rigid metal or semi-rigid plastics but may alternatively be made of substantially softer silicone rubber. Soft cups are less likely to damage a baby's head in use; conversely, relatively rigid cups are less likely to slip off and so to have to be reattached, although reattachment of a rigid cup may be difficult or impossible due to deformation of the baby's skull or scalp from the first attempt at attachment. The type of cup used may depend on the baby's position. If the baby is in a position that makes delivery more difficult, then a metal cup or a semi-rigid plastics cup may be used in preference to a soft plastics cup because the more rigid types are less likely to become dislodged.

There is also a choice of cup sizes. As a rule, the wider the cup, the more securely the cup will be held to a baby's head under a given vacuum, as sub-atmospheric pressure is applied across a greater area of the cup. Occasionally, however, the cup comes off a baby's head in use and has to be reapplied. This happens especially if the baby's head is large or in a position where the cup does not fit well.

Without trial and error, it is difficult to know whether an effective seal has been achieved between the cup and the baby's head before force is applied to pull the baby. This is important because repeated slippage of the cup increases the probability of having to resort to forceps or a caesarean section. Indeed, if the cup slips more than twice, current practice in obstetrics is to abandon use of a vacuum extractor and instead opt for another delivery method such as the use of forceps or a caesarean section.

A wide cup is also desirable because applying a given force over a greater area of the baby's head is less likely to injure the baby. For example, a baby often has a temporarily deformed head after use of a ventouse: in particular, the baby's head may be left with a lump called a chignon, and/or a blood blister, either of which may take some days to resolve.

Of course, there is a limit as to how large a cup can be before it becomes difficult to insert into the mother's vagina, risking discomfort or injury to her. In general, if a ventouse cup is small and soft so that it can be deformed to fit into the vagina, there is a fair chance that the mother will not require an episiotomy; conversely, the larger and more rigid the cup, the more likely it is that an episiotomy will be needed. There is therefore a trade-off between the efficacy of the cup and the comfort of the mother.

It is generally accepted that forceps present a greater risk of injury to the mother and baby than the use of a ventouse. For example, forceps deliveries may bruise the baby and may disturb the mother's bladder and bowel functions. Certainly, women have reported that forceps delivery causes greater discomfort both during and after birth than ventouse delivery. It is also crucial for the physician to know the orientation of the baby's head when using forceps, and forceps can be difficult to lock once in position. In contrast, vacuum extraction can be performed without exact knowledge of the orientation of the head and before the cervix is completely dilated because the suction cup takes no additional space around the baby's head. This can help a very distressed baby to be born quickly. Also, it is difficult for a physician using a ventouse to apply excessive force to a baby's head as the cup will tend to slip off in that event. Consequently, many physicians prefer using a ventouse to forceps for assisted deliveries.

Whilst the idea of using a suction cup upon the foetal head dates back to the eighteenth century, the modern vacuum extractor was invented by Malmström in 1954 and there has been considerable patent activity in that field ever since. For example, U.S. Pat. Nos. 5,019,086 and 5,810,840 disclose vacuum extractors having a rigid cup, an elongated stem and flexible means between the cup and the stem allowing the cup to be folded into an insertion position substantially parallel to the stem thereby facilitating insertion of the cup into the birth canal. However, the diameter of the cup itself cannot be reduced in this manner. By contrast, U.S. Pat. No. 5,224,947 and U.S. Pat. No. 5,569,265 are examples of flexible cups made of a soft and resilient material, the latter being in the form of a bonnet that can be rolled over the baby's cranium. More recently, an International patent application published as WO99/58071 disclosed a hand-held vacuum extractor that combines a pump and a handle into a single hand-held unit connected to a rigid vacuum cup by a tube. Advantageously, a physician can control vacuum using the pump and apply traction via the tube using one hand. This is the basis of a commercially-successful vacuum extractor known among obstetricians as the ‘Kiwi’ device, which employs a cup of standard 5 cm diameter. However, the Kiwi device is not suitable for mid-cavity use.

The Applicant's previous International patent application published as WO 2006/008532 discloses a head for an obstetric vacuum extractor that has a convex distal part reconfigurable into a concave suction cup. In the embodiments described, the head comprises single-walled distal and proximal parts, wherein the distal part can be deformed to create a double-walled suction cup in cooperation with the proximal part. Thus the head may be inserted easily into the mother's vagina, the head having a convex distal part upon insertion, whereupon the convex distal part is reconfigured into a concave suction cup for engagement with the baby's head, suction is applied to the cup to attach the cup to the baby's head, and force is applied to the baby's head via the cup to aid delivery.

U.S. Pat. No. 6,090,041 discloses a vacuum-actuated surgical retractor for retracting tissue (e.g. an organ) of a patient during surgery. It does not disclose an obstetric vacuum extractor but in some variants, the head of the retractor has a flexible end-piece. This flexibility allows the end-piece to convert between a convex shape and a concave shape in response to an application of force to the end-piece. The end-piece passively conforms to the shape of the tissue that the distal end of the retractor is pressed against during a surgical procedure, presumably to improve suction. Again, however, the overall diameter of the retractor head cannot be reduced for the purpose of insertion, at least not without a catheter for endoscopic procedures—in which case the distal part of the head is not convex when it is of reduced diameter and being inserted.

Against this background, and from one aspect, the invention resides in an obstetric vacuum extractor comprising a cylinder; a cup communicating with the cylinder; and a piston movable within the cylinder to evacuate the cup; wherein the piston is movable within the cylinder by a handle connected to the piston by an extensible bias element permitting relative movement of the handle with respect to the piston.

The bias element is capable of limiting transmission of force from the handle to the piston, to avoid excessive suction being applied to the baby's head. Also, relative movement of the handle with respect to the piston may be used to operate an indicator to display confirmation that low pressure has been maintained within the cylinder and the cup. This helps to prevent the cup slipping from the baby's head if there is an inadequate seal and hence inadequate suction to remain in place when the extractor is used to apply force to the baby.

The indicator suitably comprises an indicator element that is movable with the piston for relative movement away from the handle to display said confirmation. Preferably, the indicator element provides a display at a proximal end of the handle. The indicator element suitably moves proximally with the handle to display a warning if low pressure is not maintained within the cylinder and the cup.

A stem extending proximally from the piston may be joined to a proximal end of the bias element. In that case, the bias element may act between the stem at a proximal end of the bias element and the handle at a distal end of the bias element. The bias element is suitably a coil spring having a lumen within which the stem extends proximally from the piston; that spring is preferably pre-loaded under compression.

Preferably, the bias element acts in compression between the stem and the handle and puts the stem under tension to pull the piston proximally when the handle is moved proximally.

An indicator element to display confirmation of low pressure is suitably provided at a proximal end of the stem. Elegantly, the indicator element may be used to join the proximal end of the bias element to the proximal end of the stem.

In another aspect, the invention resides in a cup for an obstetric vacuum extractor, the cup comprising:
radially inner and outer sections, at least the outer section being reconfigurable, by applying distal force to a proximal side of the cup, from a convex insertion state for insertion into a mother's birth canal into a concave engagement state for engagement with a baby's head; and
a boundary between the inner and outer sections shaped such that the inner section may be concave while the outer section is convex.

This aspect of the invention also embraces an obstetric vacuum extractor comprising such a cup.

The inner section of the cup may have a concave profile and its outer section may have a convex profile when the cup is in an intermediate state during transition from the insertion state to the engagement state. The inner section may have a convex profile when the cup is in the insertion state.

In preferred embodiments to be described, the boundary comprises an annular step between the inner and outer sections, the step defining a distally-facing rim around the inner section when the inner section has a concave profile. There may be a corresponding step on a proximal side of the cup.

At least one bore suitably extends from the proximal side to the distal side of the cup and communicates with the inner section of the cup.

Preferably, the cup further comprises a seal formation around the outer section on its distal side. That seal formation may comprise two or more generally parallel walls extending around the outer section. Sealing and grip are enhanced if the cup is arranged to be under resilient circumferential tension when engaged with a baby's head in the engagement state.

The cup of the invention is preferably arranged to return resiliently to the engagement state, without further distal force, once pushed distally to a return position beyond an intermediate state in which the inner section is concave while the outer section is convex.

In a third aspect, the invention resides in an obstetric vacuum extractor, comprising:
a shaft;
a cup at a distal end of the shaft, the cup being reconfigurable from a convex insertion state for insertion into a mother's birth canal into a concave engagement state for engagement with a baby's head; and
a pushing member movable distally with respect to the shaft to apply distal force to a proximal side of the cup to effect said reconfiguration.

The pushing member may comprise a sleeve that extends around and is slidable with respect to the shaft.

The pushing member suitably comprises a plurality of fingers at its distal end for bearing against a proximal side of the cup to effect said reconfiguration. Neighbouring fingers may extend distally to different extents. In this way, the distal end of the pushing member, as defined by tips of the fingers, may be inclined with respect to a central longitudinal axis of the shaft.

The pushing member may expand laterally at its distal end as it pushes against a proximal side of the cup to effect said reconfiguration. For example, fingers may be resiliently or pivotably mounted to the remainder of the pushing member to move outwardly at their distal ends as they push against the proximal side of the cup.

In order that the invention may be more readily understood, reference will now be made by way of example to the accompanying drawings, in which:

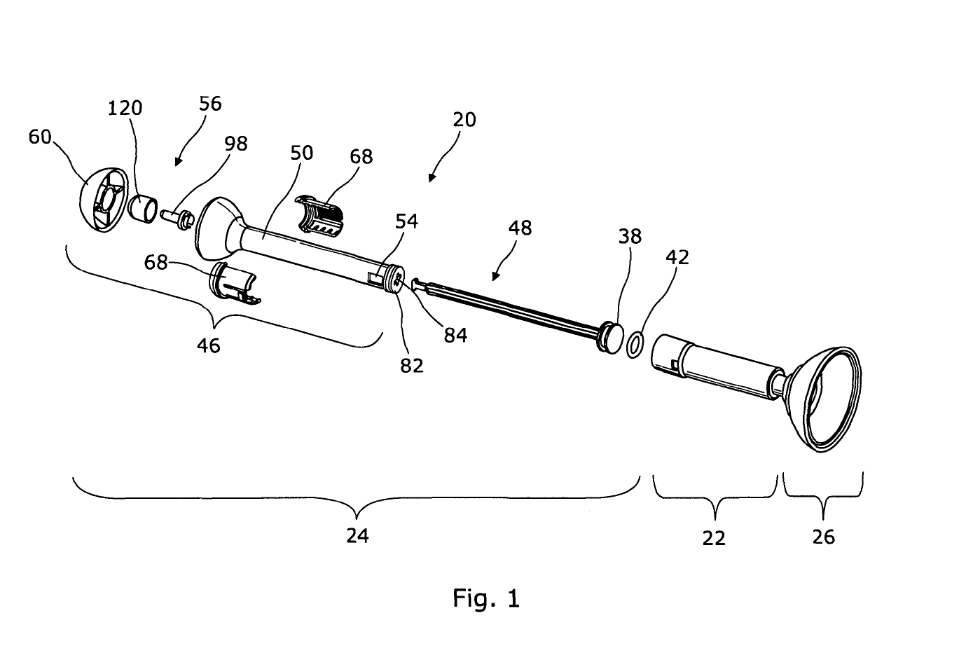

FIG. 1 is an exploded perspective view of an obstetric vacuum extractor in accordance with the invention;

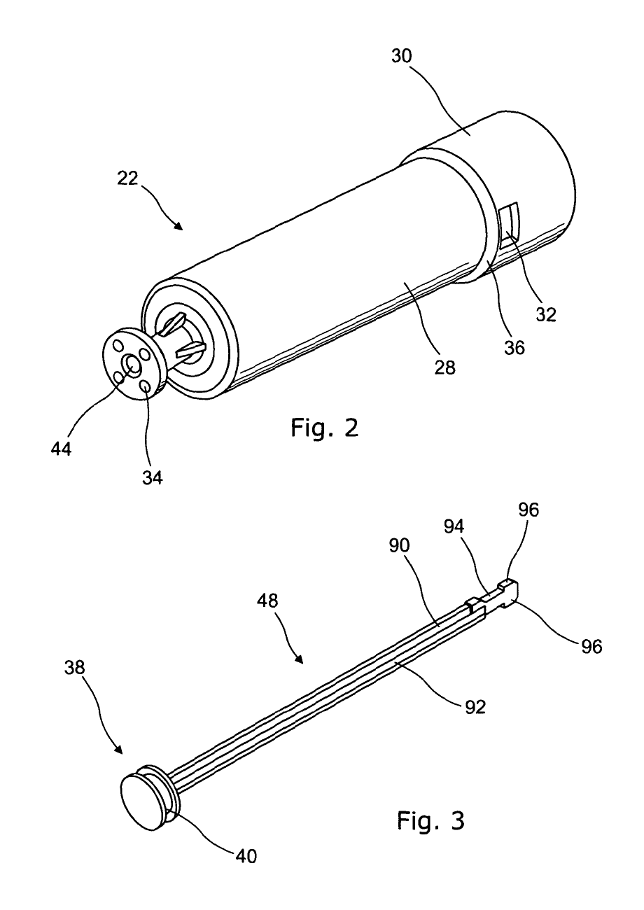

FIG. 2 is an enlarged perspective view of a barrel shown in FIG. 1;

FIG. 3 is a perspective view of a piston and stem shown in FIG. 1;

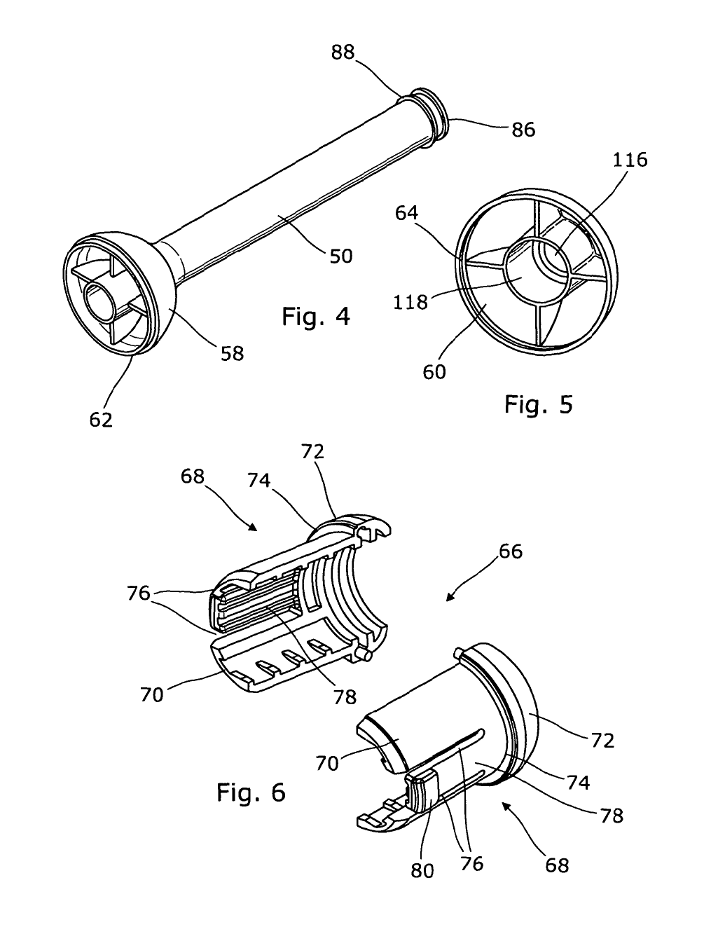

FIGS. 4 and 5 are perspective views of snap-fit handle components shown in FIG. 1;

FIG. 6 is an enlarged perspective view of a collar shown in FIG. 1;

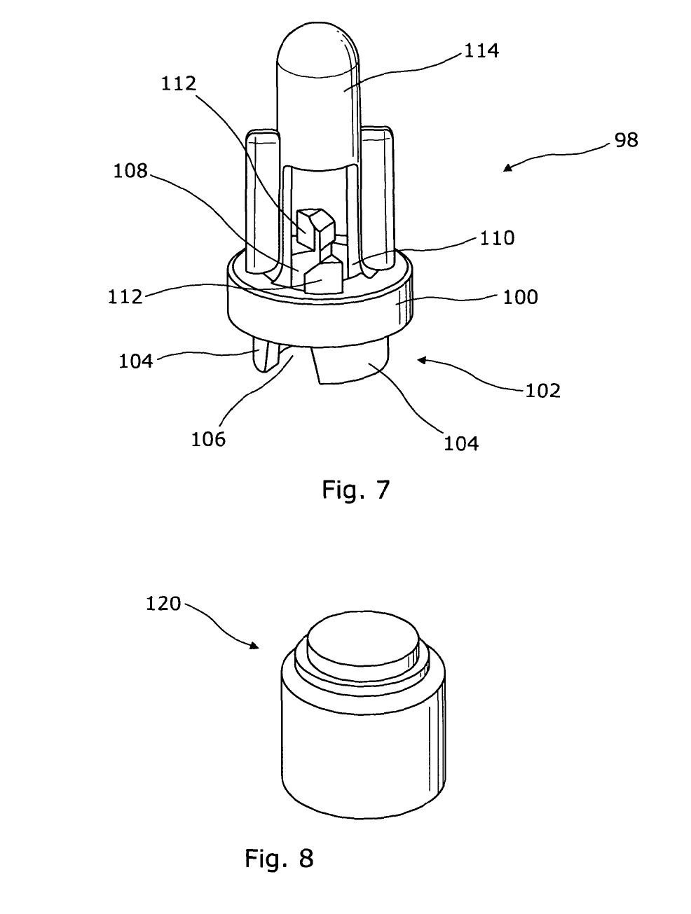

FIG. 7 is an enlarged perspective view of a spring retainer shown in FIG. 1;

FIG. 8 is an enlarged perspective view of a window element shown in FIG. 1;

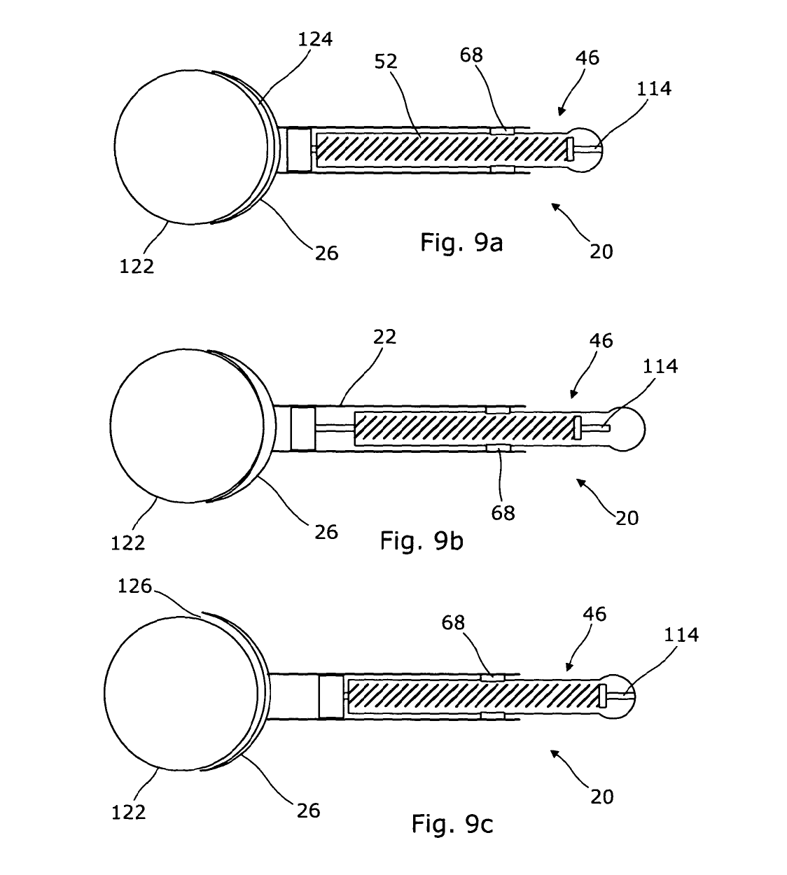

FIGS. 9a, 9b and 9c are schematic sectional side views of an extractor in accordance with the invention in use being engaged with a baby's head;

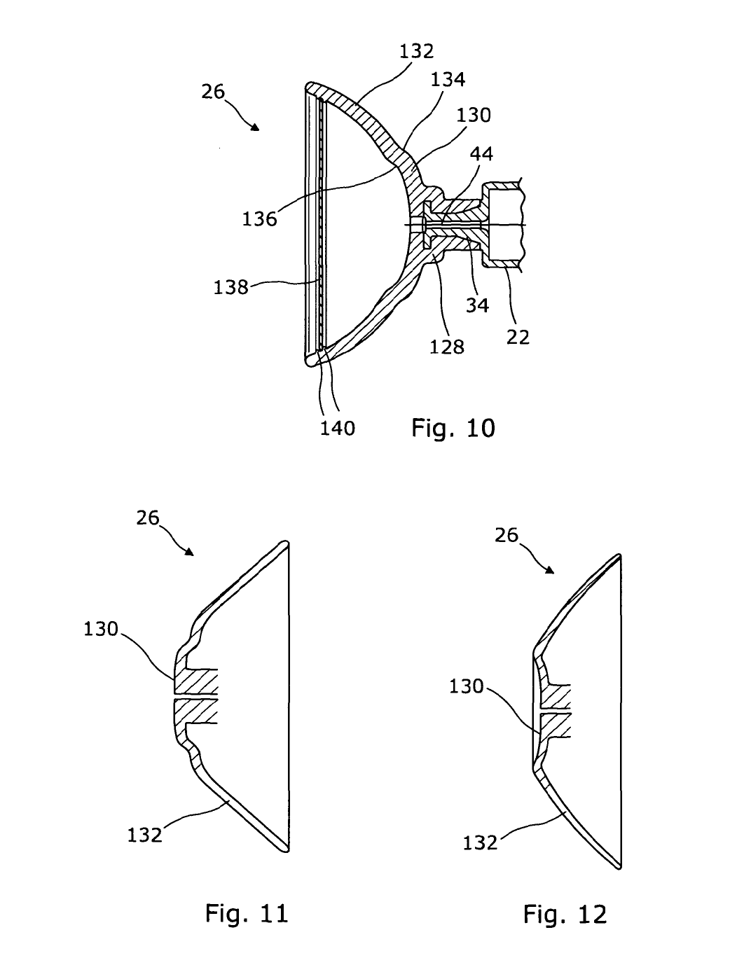

FIGS. 10, 11 and 12 are sectional side views of the cup shown in FIG. 1, with the cup being shown in concave, convex and intermediate configurations;

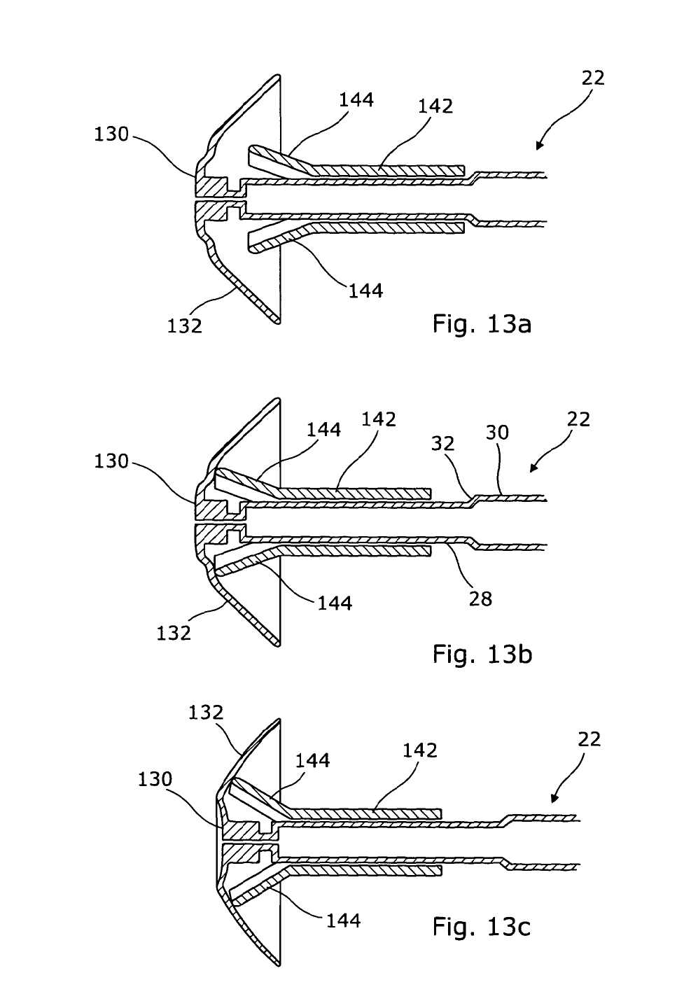

FIGS. 13a, 13b and 13c are schematic sectional side views of a second embodiment of the invention in which an optional sleeve is used to reverse the cup from convex to concave configurations after the cup has been inserted into the birth canal; and

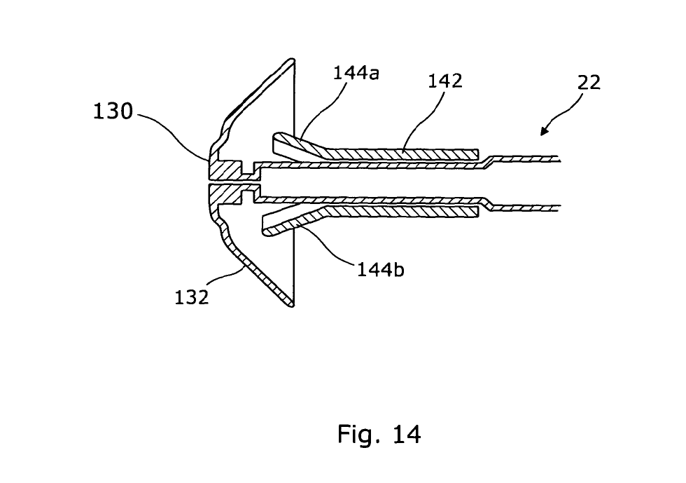

FIG. 14 is a schematic sectional side view of a variant of the second embodiment.

Referring to FIG. 1 of the drawings, a vacuum extractor 20 in accordance with the invention comprises a tubular barrel 22; an actuator rod 24 movable longitudinally with respect to the barrel 22 in telescopic fashion; and a cup 26 fixed to the distal end of the barrel 22.

As best shown in the enlarged view of FIG. 2, the barrel 22 is preferably of translucent plastics material to allow a user to view the operation of the extractor 20. The barrel 22 comprises three portions, namely: a straight-sided body portion 28; a wider proximal portion 30 whose wall is penetrated by diametrically-opposed oblong cut-outs 32; and a narrower T-section stub 34 at the distal end of the body portion 28, to which the cup 26 is attached by overmoulding. A frusto-conical circumferential shoulder 36 encircles the barrel 22 between the body portion 28 and the proximal portion 30.

A circular piston 38 at the distal end of the actuator rod 24 slides proximally within the barrel 22 to expand a chamber disposed on the distal side of the piston 38 within the barrel 22. For this purpose, the piston 38 has a circumferential groove 40 supporting an o-ring 42 as shown in exploded view in FIG. 1, for sealing sliding engagement with the internal tubular wall of the body portion 28. A lubricant may be applied to that internal wall to facilitate sealing and sliding. The chamber communicates with the interior of the cup 26 through a narrow bore 44 that extends through the distal stub 34 on its central longitudinal axis.

The actuator rod 24 is longitudinally extensible, with relative movement being possible between the piston 38 and a handle 46 of the actuator rod 24 that protrudes to a variable extent from a proximal end of the barrel 22. For this purpose, the piston 38 is attached to a stem 48 that extends proximally from the piston 38 to be received telescopically within a tubular shaft 50 of the handle 46. The piston 38 itself lies distally beyond the shaft 50.

A coil spring 52 housed within the handle 46 acts in compression between the shaft 50 of the handle 46 and the stem 48 as the handle 46 is pulled proximally with respect to the barrel 22. The coil spring 52 is omitted from FIG. 1 but is shown in the schematic views of FIGS. 9a to 9c. The stem 48 attached to the piston 38 is disposed within the lumen of the coil spring 52 inside the handle 46. Thus, a proximal pulling force applied to the handle 46 is transmitted from the handle 46 to the piston 38 via the spring 52 and the stem 48.

This arrangement enables a pressure-limiting function as the piston 38 is connected to the handle 46 via the pre-compressed spring 52, which limits the degree of vacuum that can be applied to the baby's head. The maximum force that can be applied to the piston 38 is the force conveyed through the spring 52, which is set to create a pressure differential in the cup 26 of less than about 0.9 bar. This is true even when the handle 46 reaches the end of its stroke, and means that however hard a user may pull on the handle 46, the baby's head will not be subjected to a damagingly low pressure.

The maximum pressure differential value may be adjusted as desired by tailoring the spring characteristics or the degree of pre-loading, it being noted that the maximum possible pressure differential is 1 bar.

The possibility of relative movement between the piston 38 and the handle 46 also enables the extractor 20 to warn a user of inadequate engagement between the cup 26 and a baby's head, as will be explained.

The shaft 50 of the handle 46 has diametrically-opposed openings 54 near its distal end, whose purpose will be explained. The shaft 50 terminates in a bulbous hollow proximal end 56, the shaft 50 and the bulbous proximal end 56 being rotationally symmetrical about their common central longitudinal axis. The shaft 50 and the bulbous proximal end 56 are of opaque plastics material with a spark-eroded external finish for a comfortable and secure grip

The shape of the handle 46 allows a variety of grips to be deployed: for example two different fist grips and a ‘T-Bar’ grip—in which the shaft extends between the user's fingers (usually the second and third fingers) and the bulbous end is received in the palm. This is useful to allow for different user preferences and also to allow the user's grip to change as the baby descends along the birth canal and the direction of pull changes.

Moving on now to FIGS. 4 and 5, these show that the handle 46 is in two main injection-moulded pieces. Specifically, a distal part 58 of the bulbous proximal end 56 is moulded integrally with the tubular shaft 50 as shown in FIG. 4, and a generally hemispherical proximal end-cap 60 as shown in FIG. 5 is snap-fitted to the distal part 58 to complete the bulbous proximal end 56 once the spring 52 is compressed inside the handle 46. For this purpose, the distal part 58 and the end-cap 60 have snap-fit formations comprising a proximally-extending flange 62 around the open proximal end of the distal part 58 that is received within an under-cut groove 64 around the open distal end of the end-cap 60.

As best shown in FIG. 6, a collar 66 within the proximal end of the barrel 22 centres the shaft 50 of the handle 46 within the bore of the barrel 22. The collar 66 also prevents the handle 46 being removed entirely from the barrel 22. The collar 66 consists of two identical injection-moulded plastics parts 68, each of generally semi-circular cross-section, which are brought together on assembly to encircle the shaft 50 of the handle 46 and to snap fit into the barrel 22. Together, the parts 68 of the collar 66 form a bearing within which the shaft 50 is a sliding fit for telescopic movement of the handle 46 with respect to the barrel 22.

The collar 66 is generally T-shaped in longitudinal section, comprising a tubular distal skirt 70 surmounted proximally by a laterally wider ring 72, with a circumferential step 74 between the skirt 70 and the ring 72. Two opposed pairs of parallel longitudinal slits 76 extend proximally from the distal end of the skirt 70, defining snap-fit formations in the form of opposed cantilevered prongs 78. The distal end portion of each prong 78 has an oblong tab 80 with a ramped distal end. The aforementioned openings 54 in the shaft 50 of the handle provide additional clearance for inward movement of the prongs 78 but if this additional clearance is not necessary in practice, the openings 54 may be omitted.

The distal end of the tubular shaft 50 of the handle 46 is closed by a transverse wall 82 that is penetrated by a cruciform aperture 84. The coil spring 52 bears distally against that wall 82 from within the shaft 50 when the spring 52 is compressed. The wall 82 extends laterally beyond the diameter of the shaft 50 to define a circumferential distal flange 86. A proximal flange 88 is spaced slightly from the distal flange 86. The distal and proximal flanges 86, 88 support the distal end of the handle 46 for sliding movement within the body portion of the barrel 22.

Thus, the handle 46 is supported by two longitudinally-spaced bearings: the distal and proximal flanges 86, 88 and the barrel 22 define a distal bearing for the handle 46, and the collar 66 defines a proximal bearing for the handle 46. This arrangement adds strength and precision of movement to the extractor 20 in use. It will also be apparent that the proximal flange 88 of the shaft 50 will bear against the distal end of the collar 66 when the handle 46 reaches its maximum proximal extension with respect to the barrel 22, to prevent the handle 46 being withdrawn entirely from the barrel 22.

The stem 48 shown in FIG. 3 is of cruciform cross-section, fitting within and extending through the correspondingly-shaped aperture 84 in the wall 82 at the distal end of the handle 46. More specifically, the stem 48 comprises two flat webs 90, 92 that intersect orthogonally along the central longitudinal axis of the stem 48. One of those webs 90 extends further proximally than the other web 92, defining a flat T-shaped blade 94 that terminates at its proximal end with opposed laterally-extending lugs 96.

The lugs 96 of the blade 94 engage a spring retainer 98 at the proximal end of the coil spring 52 whereby the stem 48 acts in tension to keep the coil spring 52 in compression. The spring retainer 98 is shown in enlarged view in FIG. 7. It comprises a circular flange 100 of slightly greater diameter than the external diameter of the coil spring 52, and a generally circular male formation 102 that is an interference fit within the lumen of the coil spring 52. The male formation 102 is therefore received resiliently within the lumen at the proximal end of the coil spring 52.

More specifically, the male formation 102 of the spring retainer 98 is in two arcuate sections 104 divided by cut-outs 106 aligned with a slot 108 in the flange 100, the slot 108 being sized to accommodate the blade 94 of the stem 48 and its laterally-extending lugs 96.

Recesses 110 lie behind catch formations 112 on the proximal side of the flange 100, the recesses 110 and the catch formations 112 being angularly offset with respect to the slot 108.

An integrally-moulded protrusion 114 extends proximally from the flange 100 of the spring retainer 98 on its central longitudinal axis. The protrusion 114 has a warning indicator function as will be explained.

At its central proximal extremity, the end-cap 60 of the bulbous proximal end 56 of the handle 46 shown in FIG. 5 has a hole 116 that leads to a pocket 118 disposed centrally within the end-cap 60. A tubular window element 120 shown in FIG. 8 is received within the pocket 118 to be visible through the hole 116. The window element 120 is shown in enlarged view in FIG. 8. It is injection moulded of transparent plastics material and has a central opening that receives the protrusion 114 of the spring retainer 98, such that the tip of that protrusion 114 may be seen at the proximal end of the handle 46.

On assembly of the actuator rod 24, the male formation of the spring retainer 98 is inserted into the proximal end of the coil spring 52, and the distal end of the coil spring 52 is inserted into the tubular shaft 50 of the handle 46 to lie against the wall 82 at the distal end of the shaft 50. The stem 48 carrying the piston 38 is then inserted into the distal end of the shaft 50 through the cruciform opening 84 and into the lumen of the spring 52 until the piston 38 encounters the distal end of the shaft 50. The spring 52 is then compressed until the blade 94 of the stem 48 protrudes through the slot 108 of the spring retainer 98 to the extent that the lugs 96 on the blade 94 reach the proximal side of the flange 100 of the spring retainer 98. The spring retainer 98 is then turned to engage the lugs 96 in respective recesses 110 behind the catch formations 112 on the proximal side of the flange 100. The bias of the coil spring 52 holds the stem 48 in tension and prevents the lugs 96 disengaging from the recesses 110.

The end-cap 60 with its window element 120 may then be aligned with the spring retainer 98 such that the protrusion 114 of the spring retainer 98 seats into the opening of the window element 120. The end-cap 60 is then pressed against the distal part 58 of the bulbous proximal end 56 to snap-fit those parts together to complete the bulbous proximal end 56 and to retain the compressed spring 52 safely within the handle 46.

Thus, the stem 48 is anchored at its proximal end to the proximal end of the coil spring 52. The coil spring 52 acts in compression between the proximal end of the stem 48 and the distal end of the shaft 50, putting the stem 48 in tension as the piston 38 at the distal end of the stem 48 bears proximally against the distal end of the shaft 50.

On assembly of the actuator rod 24 with the barrel 22, the two parts 68 of the collar 66 are assembled around the shaft 50 of the handle 46. As the actuator rod 24 is slid into the barrel 22 with the piston 38 in sliding sealing engagement within the body of the barrel 22, the skirt 70 of the collar 66 is inserted into the open proximal end portion of the barrel 22 with the tabs 80 angularly aligned with the cut-outs 32. Where they encounter the inner wall of the barrel 22, the tabs 80 initially force the prongs 78 to deflect inwardly; the tabs 80 then snap-fit outwardly into the cut-outs 32 to retain the collar 66 within the barrel 22. The ring 72 of the collar 66 remains outside the barrel 22, with the step 74 between the skirt 70 and the ring 72 of the collar 66 bearing against the proximal end of the barrel 22.

Moving on now to FIGS. 9a, 9b and 9c, these show schematically how the extractor 20 of the invention can warn a user of inadequate engagement between the cup 26 and a baby's head 122. As noted previously, this is important because if a cup 26 slips more than twice, current practice in obstetrics is to abandon use of a vacuum extractor and instead opt for another delivery method such as the use of forceps or a caesarean section.

FIG. 9a shows the cup 26 in its concave configuration against with the baby's head 122 but with the interstice 124 between the cup 26 and the baby's head 122 not yet evacuated. FIGS. 9b and 9c show what happens after attempts are made to evacuate the interstice 124 by pulling the handle 46 of the actuator rod 24, FIG. 9b showing successful engagement and FIG. 9c showing unsuccessful engagement due to a gap 126 around the periphery of the cup 26.

The key to the warning indicator function is that the handle 46 is movable proximally with respect to the piston 38 against the bias of the spring 52. However such relative movement between the handle 46 and the piston 38 is only possible if the piston 38 resists proximal movement by virtue of low pressure in the chamber within the barrel 22 on the distal side of the piston 38. This, in turn, is only possible if there is an adequate seal between the cup 26 and the baby's head 122 as shown in FIG. 9b. If there is an inadequate seal between the cup 26 and the baby's head 122 as shown in FIG. 9c such that a sufficiently low pressure cannot be maintained in that chamber, the piston 38 will move proximally in unison with the handle 46 by virtue of the compressive force of the spring 52 acting on the piston 38 via the stem 48.

It is in this way that the protrusion 114 on the spring retainer 98 provides the warning indicator function. On pulling the handle 46 proximally, the piston 38—and hence the stem 48 attached to the piston 38 and the spring retainer 98 engaged with the stem 48—will not move to the same extent as the handle 46 if low pressure is being maintained in the chamber as shown in FIG. 9b. The spring 52 shortens as can be seen. This, in effect, pulls the protrusion 114 of the spring retainer 98 away from the proximal end of the handle 46, so that the protrusion 114 is no longer visible in the window element 120. The user can therefore infer that a good seal has been achieved and so can start applying force to the baby's head 122 using the extractor 20.

Conversely if a sufficiently low pressure is not maintained in the chamber as the handle 46 is pulled proximally, the piston 38—and hence the stem 48 attached to the piston 38 and the spring retainer 98 engaged with the stem 48—will move proximally in unison with the handle 46 as shown in FIG. 9c. The result is that the protrusion 114 will still be visible in the window element 120 at the proximal end of the handle 46, warning the user that a further attempt must be made to achieve an adequate seal before attempting to apply force to the baby's head 122 using the extractor 20, as there is otherwise a risk that the cup 26 could slip from the baby's head 122.

The warning indicator function indicates loss of vacuum at any time after the cup 26 has been applied to the baby's head 122. This includes the period after pulling starts and not merely the period before pulling starts.

Whilst the user could possibly see relative movement between the piston 38 and the handle 46 upon looking through the translucent wall of the barrel 22, this cannot be guaranteed because the barrel 22 may well be obscured in use. The warning indicator location at the proximal end of the handle 46 is much easier for the user to see and is more reliably visible in use.

In view of its warning indicator function, it is preferred that the spring retainer 98—including the integrally moulded protrusion 114—is of a brightly-coloured plastics material that contrasts strongly with the colour of the handle 46. For example, the spring retainer 98 may be red and the handle 46 may be white.

Referring now in detail to the cup 26 and referring to FIGS. 10, 11 and 12 of the drawings, the cup 26 is integrally moulded of flexible rubber but not all sections of the cup 26 have the same degree of flexibility.

The shape of the cup 26 will firstly be described in its normal concave configuration as shown in FIG. 10, i.e. concave as viewed from the distal end of the extractor 20. In distal progression and radially-outward succession, the exterior of the cup 26 has three sections, namely: a base 128 attached by overmoulding to the distal stub 34 of the barrel 22, the base 128 being laterally wider at its distal end than at its proximal end to form a relatively rigid T-shape in longitudinal section matching the T-section of the stub 34; an inner cup section 130 whose wall curves outwardly and distally from the base 128; and an outer cup section 132 whose wall curves outwardly and distally from the inner cup section 130. An annular convex step 134 is apparent on the external or proximal surface between the inner and outer cup sections 130, 132.

The inner and outer cup sections 130, 132 each have a generally spheroidal profile of curvature that, moving distally at a constant rate, first widens relatively rapidly and then widens relatively slowly. Thus, the inclination of the external surface of the cup sections 130, 132 with respect to the central longitudinal axis of the cup 26 is initially relatively steep and thereafter becomes progressively shallower. The spheroidal profile of the inner cup section 130 is relatively steeply inclined in comparison with that of the outer cup section 132, hence being relatively oblate—i.e. a more flattened spheroid in terms of distal extension along the central longitudinal axis.

Conversely, only the inner and outer cup sections 130, 132 are visible on the internal or distal surface of the cup 26, which has an annular concave step 136 between those sections 130, 132 corresponding to the convex step 134 on the external surface of the cup 26. The bore 44 extends through the base 128 into the inner cup section 130. An integrally-moulded seal formation 138 comprises an inwardly-extending pair of parallel sealing walls 140 extending circumferentially around the interior of the outer cup section 132 near its distal extremity. If the seal of one wall 140 is compromised, the other wall 140 can maintain an effective seal to keep the cup 26 securely attached to the baby's head 122 as force is applied via the extractor 20.

The base 128 is stiffly mounted to the barrel 22 but the barrel 22 can pivot with respect to the remainder of the cup 26 by virtue of deformation in the inner cup section 130, particularly near its junction with the base 128.

The cup is sized to be about 90% of the diameter of the smallest expected baby's head 122 and therefore inherently to grip a baby's head 122 of any expected size. The design of the seal walls 140 is intended to grip the baby's scalp, with the cup 26 elastically pulling the seal walls 140 onto the skin as the cup is of slightly smaller diameter then the baby's head 122.

Moving on now to FIGS. 11 and 12, these show how the cup 26 can be inverted from distally concave to distally convex for ease of insertion into the birth canal. In this configuration, the diameter of the cup 26 can be reduced in at least one radial direction by squeezing the cup 26 laterally, for example into a oval shape, using the user's fingers to ease it into the available space. Now, as shown in FIG. 11, the distal extremity of the cup 26 is the centre of the inner cup section 130, which has become convex.

When the inner cup section 130 encounter's the baby's head, the inner cup section 130 is held against the baby's head while the outer cup section 132 is pressed distally to ease the cup 26 back into its original concave form. That reversal process takes place in two stages: firstly, the outer cup section 132 remains convex but its radially inner part pushes the radially outer part of the inner cup section 130 distally so that the inner cup section 130 becomes concave as shown in FIG. 12; only then does the second stage happen, with further distal pressure on the outer cup section 132 causing the outer cup section 132 to become concave too as the cup 26 regains the configuration shown in FIG. 10.

When the cup 26 is in the first stage with only the inner cup section 130 convex as shown in FIG. 12, the inner cup section 130 can effect initial stabilising engagement of the extractor with the baby's head 122 to locate the extractor before performing the second stage. This improves the reliability and ease of performing the second stage, which completes the application of the cup 26 to the baby's head 122. If desired, the user can evacuate the inner cup section 130 by pulling the handle 46 proximally relative to the barrel 22 before performing the second stage, being careful while pulling the handle 46 not to pull the inner cup section 130 away from the baby's head 122 before an adequate seal has been formed.

Once past an intermediate point, the resilience of the cup material pulls the outer cup section 132 quickly back into its original concave form, with limited further pressure from the user. The cup 26 therefore applies itself to the baby's head 122 semi-automatically. As the cup 26 returns to its original concave configuration shown in FIG. 10, it does so with a progressive rolling contact across the baby's head 122 that helps to expel air and to ensure a good firm engagement between the extractor 20 and the baby's head 122 at the first attempt.

The user may check the rim of the cup 26 with his or her index finger to ensure that none of the vaginal wall has become trapped between the cup 26 and the baby's head 122.

Once the cup 26 has fully engaged the baby's head 122, the handle 46 can be pulled proximally to evacuate much of what little air remains between the baby's scalp and the internal surface of the cup 26. Reference is made to FIGS. 9a to 9c in this respect. It should be noted that, predominantly, the cup 26 conforms to the shape of the baby's head 122 rather than vice versa, unlike rigid extractor cups known in the prior art that deform the baby's head by forceful suction and may leave a chignon. In the invention as described, engagement of the cup 26 with the baby's head 122 does not rely upon forceful suction but instead relies upon intimate flexible contact between the cup 26 and the scalp, with air being evacuated from the interstice 124 and ingress of air into the interstice 124 being blocked by the peripheral seal 138 of the outer cup section 132.

As noted above, the proximally-positioned warning indicator on the handle 46 allows the user to verify that an adequate seal has been established before the user attempts to apply force to the baby's head 122 via the handle 46 of the extractor 20.

Whilst both the cup 26 and the handle 46 are firmly attached to the barrel 22 for good control allowing accurate application of force to the baby's head 122, it is possible for the handle 46 to move with respect to the cup 26 in helpful ways that allow the baby to move as it negotiates the birth canal during delivery. Specifically, the baby can turn while being delivered as the cup 26 and the barrel 22 are able to turn about the central longitudinal axis of the extractor 20 with respect to the handle 46. Also, it is possible for the cup 26 to pivot about a transverse axis with respect to the handle 46 and the barrel 22.

Referring now to FIGS. 13a to 13c, these show an optional refinement of the invention. Whilst the outer section 132 of the cup 26 can be pushed distally with the user's fingers and this has been found to work well, it is also possible to provide a sleeve 142 that can be grasped by the user and pushed distally against the outer section 132 of the cup 26. This may be an easier action in a confined space and may also be more reliable and consistent than relying on the fingers alone.

The sleeve 142 is a tube that is a sliding fit on the outside of the barrel 22, being movable longitudinally between extremes defined by the cup 26 at one end and at the other end by the shoulder 36 on the barrel 22 between its body portion 28 and its enlarged proximal portion 30. The sleeve 142 has a flared distal end comprising an array of fingers 144, angularly spaced around the central longitudinal axis of the extractor 20.

In a rest position as shown in FIG. 13a, the sleeve 142 is at its proximal extremity against the shoulder 36, to provide clearance for inverting the cup 26 for insertion into the birth canal. After the inverted cup 26 has been inserted into the birth canal, the sleeve 142 is advanced distally along the barrel 22 to reverse the cup 26 back to its original shape. The first stages of that process are shown in FIGS. 13b and 13c: FIG. 13b shows the fingers 144 of the sleeve 142 having just encountered the cup 26 and FIG. 13c shows the cup 26 deformed to the intermediate configuration shown in FIG. 12.

The fingers 144 extend distally and outwardly to engage the proximal side of the reversed cup 26 at locations on the outer cup section 132, just outside the inner cup section 130 as shown in FIG. 13b. Preferably, the fingers 144 are resilient or pivotably mounted to the remainder of the sleeve 142 such that continued distal movement after encountering the cup 26 causes them to splay further apart as shown in FIG. 13c, moving their points of contact with the cup 26 radially outwardly to promote effective and consistent recovery of the cup 26 to its original concave state. All that the user needs to do is to grasp the proximal portion of the sleeve 142 and to slide it distally along the barrel 22: the proximal end of the sleeve 142 is easy to access and to manipulate. The sleeve 142 may also help the user to resist any tendency to pull the barrel 22 away from the baby's head 122 as the cup 26 is being reconfigured to its original concave shape, which could otherwise threaten the integrity of the seal.

Finally, FIG. 14 illustrates a variant of the embodiment shown in FIGS. 13a to 13c, in which some fingers 144b are longer than other fingers 144a. Thus, the fingers at the distal end of the sleeve 142 may extend distally to different extents moving around the circumference. More specifically, the fingers 144a, 144b may be of progressively increasing length from the shortest to the longest and then progressively decreasing length from the longest to the shortest moving from one finger to the next circumferentially. The effect is that the distal end of the sleeve 142, as defined by the tips of the fingers 144a, 144b, is inclined with respect to the central longitudinal axis of the barrel 22 rather than being perpendicular to that axis, as it is when the fingers 144 are all the same length as in FIGS. 13a to 13c. So, the longest fingers 144b press first on one side of the inverted cup 26 to ease it back to its original shape with less force being needed to effect the transformation. The transformation then takes place in a circumferential, gradual manner.

1. An obstetric vacuum extractor comprising a cylinder; a cup communicating with the cylinder; and a piston movable within the cylinder to evacuate the cup; wherein the piston is movable within the cylinder by a handle connected to the piston by an extensible bias element permitting relative movement of the handle with respect to the piston.

2. The extractor of claim 1, wherein a stem extends proximally from the piston and is joined to a proximal end of the bias element.

3. The extractor of claim 2, wherein the bias element acts between the stem at a proximal end of the bias element and the handle at a distal end of the bias element.

4. The extractor of claim 2, wherein the bias element acts in compression between the stem and the handle and puts the stem under tension to pull the piston proximally when the handle is moved proximally.

5. The extractor of claim 2, wherein the bias element is a coil spring having a lumen within which the stem extends proximally from the piston.

6. The extractor of claim 1, wherein the bias element is pre-loaded.

7. The extractor of claim 1, wherein the bias element limits transmission of force from the handle to the piston.

8. The extractor of claim 1, wherein said relative movement of the handle with respect to the piston operates an indicator to display confirmation that low pressure has been maintained within the cylinder and the cup.

9. The extractor of claim 8, wherein the indicator comprises an indicator element that is movable with the piston for relative movement away from the handle to display said confirmation.

10. The extractor of claim 9, wherein the indicator element moves proximally with the handle to display a warning if low pressure has not been maintained within the cylinder and the cup.

11. The extractor of claim 9, wherein the indicator element provides a display at a proximal end of the handle.

12. The extractor of claim 9, wherein: a stem extends proximally from the piston and is joined to a proximal end of the bias element; and the indicator element is at a proximal end of the stem.

13. The extractor of claim 12, wherein the indicator element joins the proximal end of the bias element to the proximal end of the stem.

14. A cup for an obstetric vacuum extractor, the cup comprising:
radially inner and outer sections, at least the outer section being reconfigurable, by applying distal force to a proximal side of the cup, from a convex insertion state for insertion into a mother's birth canal into a concave engagement state for engagement with a baby's head; and
a boundary between the inner and outer sections shaped such that the inner section may be concave while the outer section is convex.

15. The cup of claim 14, wherein the inner section has a concave profile and the outer section has a convex profile when the cup is in an intermediate state during transition from the insertion state to the engagement state.

16. The cup of claim 14, wherein the boundary comprises an annular step between the inner and outer sections, the step defining a distally-facing rim around the inner section when the inner section has a concave profile.

17. The cup of claim 16 and having a corresponding step on a proximal side of the cup.

18. The cup of claim 14, wherein the inner section has a convex profile when the cup is in the insertion state.

19. The cup of claim 14, further comprising at least one bore extending from the proximal side to the distal side of the cup and communicating with the inner section.

20. The cup of claim 14, further comprising a seal formation around the outer section on its distal side.

21. The cup of claim 20, wherein the seal formation comprises a plurality of generally parallel walls extending around the outer section.

22. The cup of claim 14 and being arranged to be under resilient circumferential tension when engaged with a baby's head in the engagement state.

23. The cup of claim 14 and being arranged to return resiliently to the engagement state, without further distal force, once pushed distally to a return position beyond an intermediate state in which the inner section is concave while the outer section is convex.

24. An obstetric vacuum extractor comprising a cup, which cup comprises:
radially inner and outer sections, at least the outer section being reconfigurable, by applying distal force to a proximal side of the cup, from a convex insertion state for insertion into a mother's birth canal into a concave engagement state for engagement with a baby's head; and
a boundary between the inner and outer sections shaped such that the inner section may be concave while the outer section is convex.

25. An obstetric vacuum extractor, comprising:
a shaft;
a cup at a distal end of the shaft, the cup being reconfigurable from a convex insertion state for insertion into a mother's birth canal into a concave engagement state for engagement with a baby's head; and
a pushing member movable distally with respect to the shaft to apply distal force to a proximal side of the cup to effect said reconfiguration.

26. The extractor of claim 25, wherein the pushing member comprises a plurality of fingers at its distal end for bearing against a proximal side of the cup to effect said reconfiguration.

27. The extractor of claim 26, wherein neighbouring fingers extend to different distal extents.

28. The extractor of claim 26, wherein the distal end of the pushing member, as defined by tips of the fingers, is inclined with respect to a central longitudinal axis of the shaft.

29. The extractor of claim 25, wherein the pushing member expands laterally at its distal end as it pushes against a proximal side of the cup to effect said reconfiguration.

30. The extractor of claim 29, wherein: the pushing member comprises a plurality of fingers at its distal end for bearing against a proximal side of the cup to effect said reconfiguration; and the fingers are resiliently or pivotably mounted to the remainder of the pushing member to move outwardly at their distal ends as they push against the proximal side of the cup.

31. The extractor of claim 25, wherein the pushing member comprises a sleeve extending around and slidable with respect to the shaft.
